(19) United States
(12) Patent Application Publication
(10) Pub. No.: US 2008/0015362 A1
(43) Pub. Date: Jan. 17, 2008

(54) Process for the preparation of optically active (S)-(+)-N,N-dimethyl-3-(1-naphthalenyloxy)-3-(2-thienyl)propanamine

(76) Inventors: Santiago Ini, Haifa (IL); Yaron Shmuely, Hedera (IL)

Correspondence Address: KENYON & KENYON LLP, ONE BROADWAY, NEW YORK, NY, 10004, US

(21) Appl. No.: 11/787,888

(22) Filed: Apr. 17, 2007

Related U.S. Application Data
(60) Provisional application No. 60/792,813, filed on Apr. 17, 2006.

(51) Int. Cl. C07D 333/16

(52) U.S. Cl. 549/4; 549/75

(57) ABSTRACT

Duloxetine intermediates, processes for their preparation, and their conversion to pharmaceutically acceptable salts of duloxetine are described.

### CROSS-REFERENCE TO RELATED APPLICATIONS

This application claims the benefit of priority to U.S. provisional application Ser. No. 60/792,813, filed Apr. 17, 2006, hereby incorporated by reference.

### FIELD OF THE INVENTION

The invention encompasses duloxetine intermediates and processes for their preparation. The invention also encompasses processes for converting the duloxetine intermediates into pharmaceutically acceptable salts of duloxetine.

### BACKGROUND OF THE INVENTION

Duloxetine is a dual reuptake inhibitor of the neurotransmitters serotonin and norepinephrine. It is used for the treatment of stress urinary incontinence (SUI), depression, and pain management. Duloxetine hydrochloride, CAS Registry No. 136434-34-9, has the chemical structure depicted in Formula I.

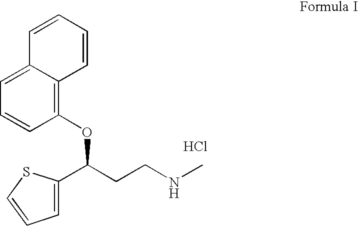

An intermediate in the synthesis of duloxetine is (S)-(+)-N,N-dimethyl-3-(1-naphthalenyloxy)-3-(2-thienyl)propanamine (“(S)-(+)-DNT”). (S)-(+)-DNT, CAS Registry No. 132335-46-7, has the chemical structure depicted in Formula II.

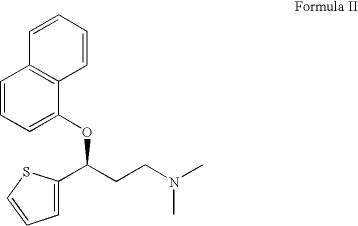

U.S. Pat. No. 4,956,388 (“'388 patent”) and U.S. Pat. No. 5,023,269 (“'269 patent”), hereby incorporated by reference, disclose a class of 3-aryloxy-3-substituted propanamines capable of inhibiting the uptake of serotonin and norepinephrine, which encompasses the compound duloxetine. The '388 and '269 patents also exemplify the synthesis of duloxetine oxalate. See '388 patent, col. 7, ll. 1-27 (example 2); '269 patent, col. 7, ll. 4-30 (example 2).

U.S. Pat. No. 5,491,243 (“'243 patent”) and U.S. Pat. No. 5,362,886 (“'886 patent”), hereby incorporated by reference, disclose a stereospecific process for the synthesis of duloxetine hydrochloride. The '886 patent describes the preparation of duloxetine by the chiral resolution of N,N-dimethyl-3-(2-thienyl)-3-hydroxypropanamine (“rac-AT-OL”) with (S)-mandelic acid (Stage a) to form (S)-(−)-N,N-dimethyl-3-(2-thienyl)-3-hydroxypropanamine (“(S)-AT-OL”), and the reaction of the (S)-AT-OL with fluoronaphthalene (Stage b) to give N,N-dimethyl-3-(1-naphthalenyloxy)-3-(2-thienyl)propanamine (“DNT”), followed by demethylation with phenyl chloroformate (Stage c), basic hydrolysis (Stage d), and acidification (Stage e) to form duloxetine hydrochloride (“DLX-HCl”). See '866 patent, col. 3, l. 26 to col. 6,l. 29. This stereospecific process of the '866 patent is illustrated in the following Scheme 1.

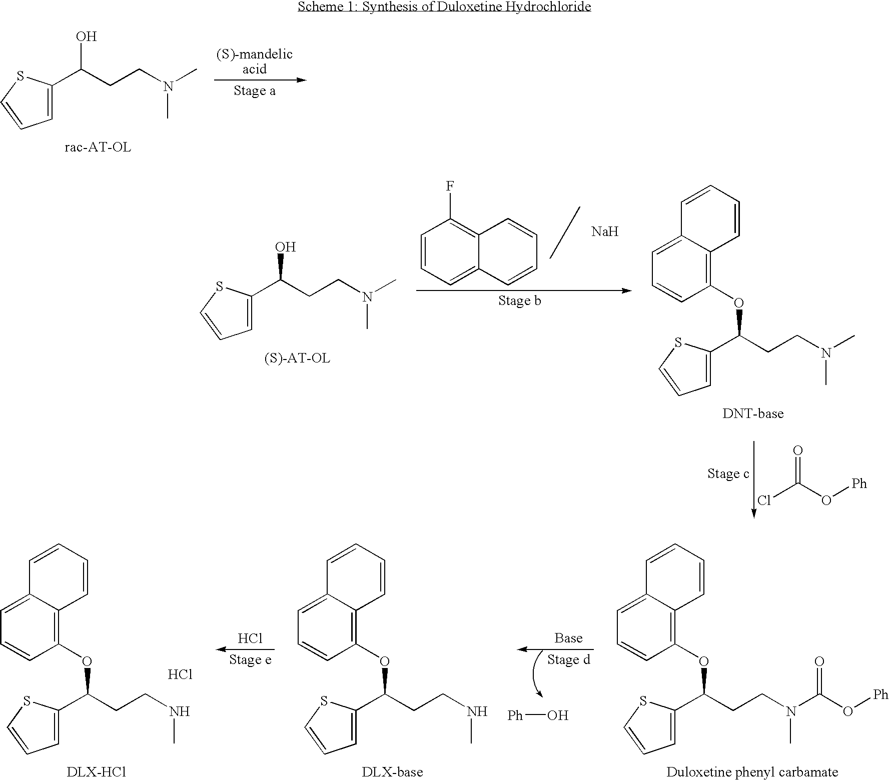

The conversion of duloxetine base to its hydrochloride salt is also described in Wheeler W. J., et al., J. Label. Cpds. Radiopharm., 36: 312 (1995) (“Wheeler article”). In both the '866 patent (stage e) and the Wheeler article, the conversion is performed in ethyl acetate.

The '886 patent and the '269 patent describe that enantiomerically pure (S)-AT-OL may be prepared by chiral resolution of rac-AT-OL.

European patent application No. 0 457 559 (“EP '559”) describes the asymmetric reduction of 3-dimethylamino-1-(2-thienyl)-1-propanone (“AT-ONE”) to (S)-AT-OL, in the presence of lithium aluminum hydride at a temperature of −75° C. See EP '559, p. 5, 1. 50 to p. 6, 1. 44. EP '559 reports that 171.6 grams of (S)-AT-OL are recovered, which corresponds to about 59% yield. Catalytic asymmetric hydrogenation of AT-ONE, in the presence of transition metal chiral complexes is described in International publication WO 2004/031168 (“WO '168”) and in U.S. publication No. 2005/0197503 (“'503 publication”). See WO '168, p. 15, 1. 29 to p. 16, 1. 8 (example 3); '503 publication, p. 4, ¶¶ 58-60 (example 5). WO '168 discloses that the (S)-AT-OL is obtained in 70% yield (67.5% enantiomeric excess) and the '503 publication discloses that the (S)-AT-OL is obtained in 79% yield (94% enantiomeric excess). International publication WO 2004/344067 describes the use of an enzymatic process.

The above-described processes for preparing (S)-AT-OL require low temperatures (such as −75° C.), expensive ligands, and/or expensive transition metals (such as ruthenium, rhodium, and iridium). The processes also employ special equipment and conditions. Accordingly, these processes are undesirable for use on an industrial scale.

Alternatively, chiral secondary alcohols have been prepared by asymmetric hydrogenation using oxazaborolidine catalysts. See Itsuno, et al., J. Chem. Soc., Chem. Comm., 315 (1981). One such oxazaborolidine catalyst is CBS (proline oxazaborolidine). See Corey, et al., J. Am. Chem. Soc., 109: 5551 (1987). The oxazaborolidine catalysts can be acquired from any source, such as Aldrich Chemicals, or can be prepared by any method known in the art, such as that described by Corey, et al., Angew. Chem. Int. Ed., 37: 1986 (1998) and illustrated in the following Scheme 2. 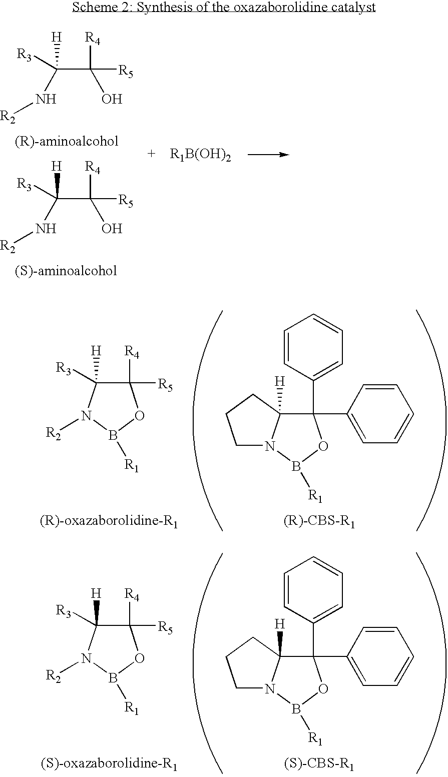
wherein R₁ can be hydrogen, C₁₋₈ alkyl, C₆₋₁₇ aryl or C₇₋₁₉ aralkyl group, and R₂₋₅ can be hydrogen, C1-8 alkyl, C3-8 cycloalkyl, C6-17 aryl, or C7-19 aralkyl group.

The enantioselective oxazaborolidine reduction of ketones containing a nitrogen atom to form an amino alcohol-borane complex was first described by Quallich, et al., Tetrahedron Letters, 34: 785 (1993) (“Quallich article”) and was described more recently by Zhang, et al., Chin. J. Chem., 19: 1130 (2001) (“Zhang article”). The Quallich article does not describe the method used to obtain the optically active amino alcohol from the amino alcohol-borane complex. In addition, the treatment employed in the Zhang article may result in the racemization of AT-OL.

Processes for the preparation of optically active intermediates of duloxetine catalyzed by CBS are disclosed in the Wheeler article and in International publication WO 03/070720 (“WO '720”). The Wheeler article describes the performance of the asymmetric reduction on a chlorine derivative, and in WO '720, the reduction is performed on a benzoyl derivative. In both publications, the asymmetric reduction must be followed by another step (either to remove the chlorine derivative or to remove the benzoyl derivative-respectively), in order to obtain the duloxetine intermediate DNT.

There is a need in the art for improved and more efficient methods for the preparation of enantiomerically pure DNT.

### SUMMARY OF THE INVENTION

In one embodiment, the invention encompasses (S)—N,N-Dimethyl-3-(2-thienyl)-3-hydroxypropanamine borane of the following structure:

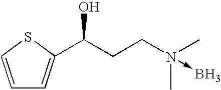

In another embodiment, the invention encompasses (R)—N,N-Dimethyl-3-(2-thienyl)-3-hydroxypropanamine borane of the following structure:

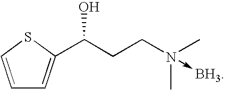

In another embodiment, the invention encompasses a process for preparing N,N-dimethyl-3-(2-thienyl)-3-hydroxypropanamine borane comprising: (a) combining 3-dimethylamino-1-(2-thienyl)-1-propanone, at least one polar aprotic solvent, a chiral oxazaborolidine catalyst and BH₃ to obtain a mixture containing N,N-dimethyl-3-(2-thienyl)-3-hydroxypropanamine borane; and (b) recovering the N,N-dimethyl-3-(2-thienyl)-3-hydroxypropanamine borane from the mixture.

In another embodiment, the invention encompasses a process for preparing duloxetine or a pharmaceutically acceptable salt of duloxetine comprising: (a) preparing (S)—N,N-dimethyl-3-(2-thienyl)-3-hydroxypropanamine borane by the above-described process; and (b) converting the (S)—N,N-dimethyl-3-(2-thienyl)-3-hydroxypropanamine borane into duloxetine or a pharmaceutically acceptable salt of duloxetine.

In another embodiment, the invention encompasses a process for preparing N,N-dimethyl-3-(1-naphthalenyloxy)-3-(2-thienyl)propanamine or a salt thereof comprising: (a) combining a base, N,N-dimethyl-3-(2-thienyl)-3-hydroxypropanamine borane, and at least one polar aprotic solvent to obtain a solution; (b) combining the solution with a 1-halonaphthalene to obtain a mixture; (c) heating the mixture to obtain N,N-dimethyl-3-(1-naphthalenyloxy)-3-(2-thienyl)propanamine; and, optionally (d) converting the N,N-dimethyl-3-(1-naphthalenyloxy)-3-(2-thienyl)propanamine to a salt thereof.

In another embodiment, the invention encompasses a process for preparing N,N-dimethyl-3-(1-naphthalenyloxy)-3-(2-thienyl)propanamine maleate comprising: (a) combining a base, N,N-dimethyl-3-(2-thienyl)-3-hydroxypropanamine borane, and at least one polar aprotic solvent to obtain a solution; (b) combining the solution with a 1-halonaphthalene to obtain a mixture; (c) heating the mixture to obtain N,N-dimethyl-3-(1-naphthalenyloxy)-3-(2-thienyl)propanamine; and (d) converting the N,N-dimethyl-3-(1-naphthalenyloxy)-3-(2-thienyl)propanamine to N,N-dimethyl-3-(1-naphthalenyloxy)-3-(2-thienyl)propanamine maleate.

In another embodiment, the invention encompasses a one-pot process for preparing N,N-dimethyl-3-(1-naphthalenyloxy)-3-(2-thienyl)propanamine or a salt thereof comprising: (a) combining 3-dimethylamino-1-(2-thienyl)-1-propanone, at least one polar aprotic solvent, a chiral oxazaborolidine catalyst and BH₃ to obtain a first mixture containing N,N-dimethyl-3-(2-thienyl)-3-hydroxypropanamine borane; (b) combining the first mixture with a base and at least one polar aprotic solvent to obtain a solution; (c) combining the solution with a 1-halonaphthalene to obtain a second mixture; (d) heating the second mixture to obtain N,N-dimethyl-3-(1-naphthalenyloxy)-3-(2-thienyl)propanamine; and, optionally (e) converting the N,N-dimethyl-3-(1-naphthalenyloxy)-3-(2-thienyl)propanamine to a salt thereof.

In another embodiment, the invention encompasses a one-pot process for preparing N,N-dimethyl-3-(1-naphthalenyloxy)-3-(2-thienyl)propanamine maleate comprising: (a) combining 3-dimethylamino-1-(2-thienyl)-1-propanone, at least one polar aprotic solvent, a chiral oxazaborolidine catalyst and BH₃ to obtain a first mixture containing N,N-dimethyl-3-(2-thienyl)-3-hydroxypropanamine borane; (b) combining the first mixture with a base and at least one polar aprotic solvent to obtain a solution; (c) combining the solution with a 1-halonaphthalene to obtain a second mixture; (d) heating the second mixture to obtain N,N-dimethyl-3-(1-naphthalenyloxy)-3-(2-thienyl)propanamine; and (e) converting the N,N-dimethyl-3-(1-naphthalenyloxy)-3-(2-thienyl)propanamine to N,N-dimethyl-3-(1-naphthalenyloxy)-3-(2-thienyl)propanamine maleate.

### DETAILED DESCRIPTION OF THE INVENTION

The invention addresses the above-described shortcomings of the prior art by providing an improved process for preparing the optically-active duloxetine intermediate N,N-dimethyl-3-(1-naphthalenyloxy)-3-(2-thienyl)propanamine (“DNT”).

As used herein, unless otherwise defined, the term “room temperature” refers to a temperature of about 15° C. to about 30° C.

The invention encompasses a process for preparing chiral DNT or salts thereof by catalytic asymmetric reduction of 3-dimethylamino-1-(2-thienyl)-1-propanone (“AT-ONE”) to N,N-dimethyl-3-(2-thienyl)-3-hydroxypropanamine (“AT-OL”) through a borane intermediate, N,N-dimethyl-3-(2-thienyl)-3-hydroxypropanamine borane (“(AT-OL)BH₃”), followed by reaction with a 1-halonaphthalene. A preferred process, resulting in the maleate salt of DNT is illustrated in Scheme 3.

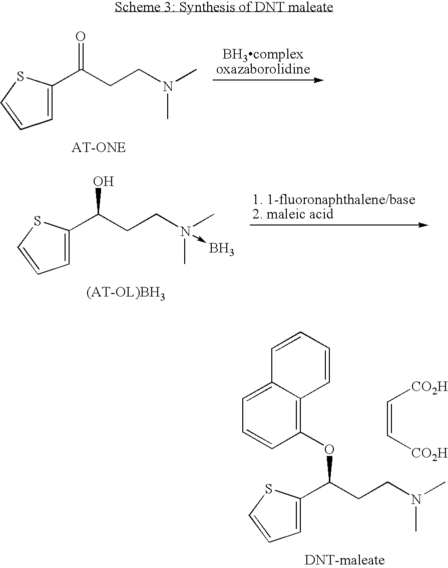

The AT-ONE is converted into (AT-OL)BH₃ by a process comprising: (a) combining AT-ONE, at least one polar aprotic solvent, a chiral oxazaborolidine catalyst and BH₃ to obtain a mixture containing (AT-OL)BH₃; and (b) recovering the (AT-OL)BH₃ from the mixture.

Typically, the polar aprotic solvent is a C₂₋₈ aliphatic or cyclic ether or aromatic hydrocarbon. Preferably, the polar aprotic solvent is toluene and more preferably tetrahydrofuran (“THF”).

The borane (BH₃) may be added as diborane dimer (B₂H₆) or a complex with a Lewis base. Preferably, the complex with the Lewis base is a borane tetrahydrofuran complex, borane 1,2-bis(tert-butylthio)ethane complex, borane 1,4-oxathiane complex, borane 4-methylmorpholine complex, borane ammonia complex, borane dimethyl sulfide complex, borane dimethylamine complex, borane diphenylphosphine complex, borane isoamylsulfide complex, borane morpholine complex, borane-morpholine, borane N,N-diethylaniline complex, borane N,N, diisopropylethylamine complex, borane pyridine complex, borane tert-butylamine complex, borane triethylamine complex, borane trimethylamine complex, or borane triphenylphosphine complex. Preferably, the BH₃ is added as a mixture in the polar aprotic solvent, more preferably THF. Preferably, the BH₃ is added in an amount of about 2 mole equivalents relative to the AT-ONE.

Typically, the mixture is maintained at about room temperature. Typically, the mixture is maintained for at least about 5 minutes to obtain the (AT-OL)BH₃. Preferably, the mixture is maintained, while stirring for about 20 minutes to about 5 hours, more preferably, for about one hour.

The obtained (AT-OL)BH₃ may be recovered by any method known to one of ordinary skill in the art. Such methods include, but are not limited to washing and evaporating the solvent under reduced pressure.

When the chiral oxazaborolidine catalyst used is an (R)-oxazaborolidine catalyst, the obtained product is (S)—N,N-dimethyl-3-(2-thienyl)-3-hydroxypropanamine borane (“(S)-(AT-OL)BH₃”). Alternatively, when the chiral oxazaborolidine used is an (S)-oxazaborolidine catalyst, the obtained product is (R)—N,N-dimethyl-3-(2-thienyl)-3-hydroxypropanamine borane (“(R)-(AT-OL)BH₃”).

(S)-(AT-OL)BH₃ has the chemical formula C₉H₁₈BNOS and the following chemical structure:

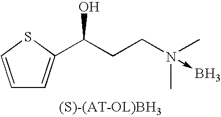

(R)-(AT-OL)BH₃ has the chemical formula C₉H₁₈BNOS and the following chemical structure:

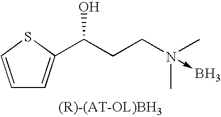

Both (S)-(AT-OL)BH₃ and (R)-(AT-OL)BH₃ are characterized by data selected from: ¹H NMR (400 MHz, DMSO d6) δ(ppm): 7.31 (t, J=6.0 Hz, 1H), 6.95 (m, 2H), 5.74 (d, J=4.0 Hz, 1H), 4.81 (m, 1H), 2.71 (m, 2H), 2.45 (s, 6H), 2.03 (m, 2H), 1.07-1.80 (m, 3H); and ¹³C NMR (100 MHz): δ 150.2, 126.8, 124.4, 123.1, 66.9, 60.9, 51.1, 50.9, 33.7.

The (S)-(AT-OL)BH₃ obtained by the above-described process may subsequently be converted into DNT or a salt of DNT, and further to duloxetine or a pharmaceutically acceptable salt of duloxetine. Salts of DNT include, but are not limited to, hydrochloride, oxalate, phosphate, succinate, fumarate, benzenesulfonate, maleate, and tartarate salts. Pharmaceutically acceptable salts of duloxetine include, but are not limited to, hydrochloride, oxalate, phosphate, succinate, fumarate, benzenesulfonate, maleate, and tartarate salts. Preferably, the pharmaceutically acceptable salt of duloxetine is a hydrochloride salt.

(AT-OL)BH₃ may be converted into DNT or a salt of DNT by a process comprising: (a) combining a base, (AT-OL)BH₃, and at least one polar aprotic solvent to obtain a solution; (b) combining the solution with a 1-halonaphthalene to obtain a mixture; (c) heating the mixture to obtain DNT; and, optionally (d) converting the DNT to a salt of DNT.

In a specific embodiment the (AT-OL)BH₃ is converted into N,N-dimethyl-3-(1-naphthalenyloxy)-3-(2-thienyl)propanamine maleate (“DNT-maleate”) by a process comprising: (a) combining a base, (AT-OL)BH₃, and at least one polar aprotic solvent to obtain a solution; (b) combining the solution with a 1-halonaphthalene to obtain a mixture; (c) heating the mixture to obtain DNT; and (d) converting the DNT to DNT-maleate.

The (AT-OL)BH₃ used as the starting material may be either the (R)— or (S)-enantiomer, to yield either (R)-DNT-maleate or (S)-DNT-maleate, respectively.

Typically, the base is a strong base. Preferably, the strong base is an alkali metal hydroxide or alkali metal alkoxide. More preferably, the strong base is potassium hydroxide (KOH), sodium methoxide, or sodium hydroxide (NaOH). The strong base may be added portionwise in order to increase the chemical yield of the DNT-maleate.

Typically, the polar aprotic solvent is a C₅-C₈ aromatic hydrocarbon, ionic liquid, dimethyl sulfoxide (“DMSO”), dimethylformamide (“DMF”), dimethylacetamide (“DMA”), acetonitrile, sulfolane, nitromethane or propylene carbonate. Preferred C₅-C₈ aromatic hydrocarbons include toluene and xylene. As used herein the term “ionic liquid” refers to salts whose melting point is relatively low (below about 100° C.). In particular, the salts that are liquid at room temperature and are called room temperature ionic liquids (“RTILs”). Preferred ionic liquids include alkylammonium halides, alkylphosphonium halides, N-alkylpyridinium halides, N-N-dialkylimidazolium halides, tetraalkylammonium tetraalkylborides, 1-alkyl-3-methylimidazolium trifluoromethanesulfonate salts, monoalkylammonium nitrate salts, halogenaluminate, chlorocuprate and 1-butyl-3-methylimidazolium tetrafluoroborate. More preferably, the ionic liquid is 1-butyl-3-methylimidazolium tetrafluoroborate. Most preferably, the polar aprotic solvent is DMA or DMSO.

Typically, the solution is provided at a temperature of from about 15° C. to about the reflux temperature of the polar aprotic solvent.

Preferably, the 1-halonaphthalene is 1-fluoronaphthalene or 1-chloronaphthalene. Preferably, before adding the 1-halonaphthalene, the mixture is cooled to room temperature.

Typically, the mixture is heated to a temperature of about room temperature to about the reflux temperature of the polar aprotic solvent to obtain DNT.

The DNT may be converted to DNT-maleate by a process comprising combining DNT and maleic acid to obtain DNT-maleate.

In one embodiment, the process comprises combining with maleic acid a solution of DNT in at least one solvent to obtain a precipitate of DNT-maleate; and recovering the DNT-maleate. The maleic acid may be either added as a solid or as a solution or suspension in an organic solvent. The solvent is preferably selected from C₁₋₈ alcohols, C₃₋₇ esters, C₃₋₈ ethers, C₃₋₇ ketones, C₆₋₁₂ aromatic hydrocarbons, acetonitrile, and water. More preferably, the solvent is acetone, n-butanol, ethyl acetate, methyl tert-butyl ether, toluene or water. Most preferably, the solvent is ethyl acetate, acetone, or n-butanol.

Typically, the combination of DNT, maleic acid, and solvent is heated. Preferably, the combination is heated to about reflux temperature of the solvent. Preferably, the combination is maintained, while heating, for about 15 minutes.

Preferably, the combination is cooled to induce precipitation of the DNT-maleate. More preferably, the combination is cooled to a temperature of about 15° C. Preferably, the combination is maintained, while cooled, for about 20 minutes to about 5 days to induce precipitation of the DNT-maleate.

The DNT-maleate may be recovered by any method known to one of ordinary skill in the art. Such methods include, but are not limited to, separating the phases, and concentrating the organic phase until a dry residue is formed. Prior to separation, the DNT-maleate may be washed in order to remove inorganic impurities, or organic impurities that are miscible in water.

The DNT-maleate prepared by the above process is obtained in high enantiomeric excess. Preferably, when (S)-DNT-maleate is prepared by the above process, it contains less than about 5% of (R)-DNT-maleate, more preferably less than about 2% of (R)-DNT-maleate, and most preferably about 1% of (R)-DNT-maleate.

Alternatively, DNT or a salt of DNT may be prepared directly from AT-ONE in a one-pot process comprising: (a) combining AT-ONE, at least one polar aprotic solvent, a chiral oxazaborolidine catalyst and BH₃ to obtain a first mixture containing (AT-OL)BH₃; (b) combining the first mixture with a base and at least one polar aprotic solvent to obtain a solution; (c) combining the solution with a 1-halonaphthalene to obtain a second mixture; (d) heating the second mixture to obtain DNT; and, optionally (e) converting the DNT to a salt of DNT.

In a specific embodiment, DNT-maleate is prepared directly from AT-ONE in a one-pot process comprising: (a) combining AT-ONE, at least one polar aprotic solvent, a chiral oxazaborolidine catalyst and BH₃ to obtain a first mixture containing (AT-OL)BH₃; (b) combining the first mixture with a base and at least one polar aprotic solvent to obtain a solution; (c) combining the solution with a 1-halonaphthalene to obtain a second mixture; (d) heating the second mixture to obtain DNT; and (e) converting the DNT to DNT-maleate.

The polar aprotic solvent of step (a) may be the same as or different than the polar aprotic solvent of step (b).

The DNT-maleate prepared by the above-described processes may be converted into duloxetine or a pharmaceutically acceptable salt of duloxetine. Pharmaceutically acceptable salts of duloxetine include, but are not limited to, hydrochloride, oxalate, phosphate, succinate, fumarate, benzenesulfonate, maleate, and tartarate salts. Preferably, the pharmaceutically acceptable salt of duloxetine is an HCl salt. The conversion of DNT to a pharmaceutically acceptable salt of duloxetine may be performed by any method known to one of ordinary skill in the art, such as the one described in the '269 patent or in U.S. publication No. 2006/0194869, for making duloxetine HCl. Preferably, the conversion is performed by a process comprising: dissolving DNT in an organic solvent to obtain a solution; combining the solution with an alkyl haloformate to obtain duloxetine alkyl carbamate; combining the duloxetine alkyl carbamate with an organic solvent and a base to obtain duloxetine; and converting the duloxetine into a pharmaceutically acceptable salt. More preferably, the conversion is performed by a process comprising dissolving DNT in a water immiscible organic solvent to obtain a first solution; adding alkyl chloroformate to the first solution at a temperature of about 5° C. to less than about 80° C. to obtain duloxetine alkyl carbamate; combining the duloxetine alkyl carbamate with an organic solvent and a base to obtain a first mixture; heating the first mixture to reflux temperature and maintaining the first mixture at reflux temperature for at least 1 to 3 hours; cooling the first mixture and adding water and an additional amount of an organic solvent to the first mixture to obtain duloxetine; recovering the duloxetine from the first mixture; combining the duloxetine with a solvent to obtain a second solution; adding hydrochloric acid to the second solution until a second mixture having a pH of about 3 to about 4 is obtained; maintaining the second mixture to obtain a solid residue; and recovering duloxetine HCl from the residue.

Having thus described the invention with reference to particular preferred embodiments and illustrative examples, those in the art may appreciate modifications to the invention as described and illustrated that do not depart from the spirit and scope of the invention as disclosed in the specification. The following examples are set forth to aid in understanding the invention but are not intended to, and should not be construed to limit its scope in any way. The examples do not include detailed descriptions of conventional methods. Such methods are well known to those of ordinary skill in the art and are described in numerous publications.

### EXAMPLES

HPLC method for measuring enantiomeric purity of DNT maleate:

| | |
|---|---|
| Column: | Daicel Chiralcel OD, 10 μm, 250 × 4.6 mm |
| Eluent: | 970 mL Hexane; 30 mL Isopropanol; 2 mL |
| | Diethylamine |
| Sample volume: | 100 μL |
| Flow: | 0.8 mL/min |
| Detector: | 230 nm |
| Column temperature: | 30° C. |
| Sample concentration: | 0.02 mg/mL |

### Example 1

### Preparation of (AT-OL)BH₃

A dried three neck flask was charged with 1.51 g of (R)-Tetrahydro-1-methyl-3,3-diphenyl-1H,3H-pyrrolo[1,2-c][1,3,2]oxazaborole [(R)-CBS-Me] and 10 ml of dry THF, and stirred under a nitrogen atmosphere. 111 ml of 1M BH₃-THF in THF and AT-ONE (10 g) were added simultaneously from two different addition funnels over a period of 30 minutes. After stirring the mixture for an additional hour, 50 ml of methanol were added in two portions and the solvent evaporated under reduced pressure to give 12.23 g of viscous oil.

### Example 2

### Preparation of DNT

A 150 ml round bottom flask equipped with mechanical stirrer, thermometer, and condenser was charged with 5 g of the (AT-OL)BH₃ complex prepared in Example 1 and 30 ml DMSO at 20° C. The mixture was stirred until complete dissolution, and 5.4 g of KOH were added and stirred for an additional time. After 30 minutes 4 ml of 1-fluoronaphthalene were added and the solution heated to 60° C. and stirred for 24 hours at the same temperature.

To the reaction mixture was added 25 ml of water followed by 4 ml HCl [5%] and 25 ml ethyl acetate. After phase separation, the water phase was extracted with ethyl acetate and the organic extracts were combined, washed with brine, and concentrated to dryness at 45° C. to give 8.37 g of brownish oil.

### Example 3

### Preparation of DNT-Maleate

To a solution of 8.11 g of the crude DNT prepared in Example 2 in 80 ml ethyl acetate, was added 3.22 g of maleic acid and stirred at reflux for 15 minutes, cooled to room temperature, and half of the volume evaporated at reduced pressure. The resulting solution was placed on an ice bath until precipitation occurred. The resulting solid was filtered off and washed with cool ethyl acetate and dried in a vacuum oven at room temperature overnight, resulting in 3.97 g of DNT-maleate (overall chemical yield: 36%, enantiomer R: 1.01%).

### Example 4

### Preparation of (AT-OL)BH₃

A dried three neck flask was charged with 1.90 g of (R)-Tetrahydro-1-phenyl-3,3-diphenyl-1H,3H-pyrrolo[1,2-c][1,3,2]oxazaborole [(R)-CBS-Ph] and 10 ml of dry THF, and stirred under a nitrogen atmosphere. 111 ml of 1M BH₃-THF in THF and AT-ONE (10 g) were added simultaneously from two different addition funnels over a period of 30 minutes.

After stirring the mixture for an additional hour, 30 ml of methanol were added dropwise while cooling in an ice bath, and the solvent was evaporated under reduced pressure to give 13.42 g of viscous oil.

### Example 5

### Preparation of DNT

A 150 ml round bottom flask equipped with mechanical stirrer, thermometer, and condenser was charged with 12.3 g of the (AT-OL)BH₃ complex prepared in Example 4 and 80 ml DMSO at 20° C. The mixture was stirred until complete dissolution, and 11 g of KOH were added and stirred for an additional time. After 30 minutes, 8 ml of 1-fluoronaphthalene were added, and the solution was heated to 60° C. and stirred for 26 hours at the same temperature.

To the reaction mixture was added 50 ml of water followed by 10 ml HCl [5%] and 60 ml ethyl acetate. After phase separation, the water phase was extracted with ethyl acetate, and the organic extracts were combined, washed with brine, and concentrated to dryness at 45° C. to give 19.11 g of dark oil.

### Example 6

### Preparation of DNT-Maleate

To a solution of 19.11 g of the DNT prepared in Example 5 in 160 ml ethyl acetate, was added 7.32 g of maleic acid. The mixture was stirred at reflux for 15 minutes, and cooled to room temperature until precipitation occurred. The resulting solid was filtered off, washed with cool ethyl acetate, and dried in a vacuum oven at 40° C. for overnight, resulting in 8.85 g of DNT-maleate (overall chemical yield: 40%).

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

We claim:

1. (S)—N,N-Dimethyl-3-(2-thienyl)-3-hydroxypropanamine borane of the following structure:

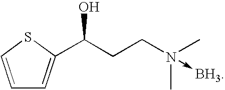

2. The (S)—N,N-dimethyl-3-(2-thienyl)-3-hydroxypropanamine borane of claim 1, characterized by data selected from: ¹H NMR (400 MHz, DMSO d6) δ(ppm): 7.31 (t, J=6.0 Hz, 1H), 6.95 (m, 2H), 5.74 (d, J=4.0 Hz, 1H), 4.81 (m, 1H), 2.71 (m, 2H), 2.45 (s, 6H), 2.03 (m, 2H), 1.07-1.80 (m, 3H); and ¹³C NMR (100 MHz): δ 150.2, 126.8, 124.4, 123.1, 66.9, 60.9, 51.1, 50.9, 33.7.

3. (R)—N,N-Dimethyl-3-(2-thienyl)-3-hydroxypropanamine borane of the following structure:

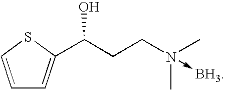

4. The (R)—N,N-dimethyl-3-(2-thienyl)-3-hydroxypropanamine borane of claim 3, characterized by data selected from: ¹H NMR (400 MHz, DMSO d6) δ(ppm): 7.31 (t, J=6.0 Hz, 1H), 6.95 (m, 2H), 5.74 (d, J=4.0 Hz, 1H), 4.81 (m, 1H), 2.71 (m, 2H), 2.45 (s, 6H), 2.03 (m, 2H), 1.07-1.80 (m, 3H); and ¹³C NMR (100 MHz): δ 150.2, 126.8, 124.4, 123.1, 66.9, 60.9, 51.1, 50.9, 33.7.

5. A process for preparing N,N-dimethyl-3-(2-thienyl)-3-hydroxypropanamine borane comprising:
(a) combining 3-dimethylamino-1-(2-thienyl)-1-propanone, at least one polar aprotic solvent, a chiral oxazaborolidine catalyst and BH₃ to obtain a mixture containing N,N-dimethyl-3-(2-thienyl)-3-hydroxypropanamine borane; and
(b) recovering the N,N-dimethyl-3-(2-thienyl)-3-hydroxypropanamine borane from the mixture.

6. The process of claim 5, wherein the polar aprotic solvent is a C₂₋₈ aliphatic or cyclic ether or aromatic hydrocarbon.

7. The process of claim 6, wherein the aromatic hydrocarbon is toluene.

8. The process of claim 6, wherein the C₂₋₈ cyclic ether is tetrahydrofuran.

9. The process of claim 5, wherein the BH₃ is present in an amount of about 2 mole equivalents relative to the amount of the 3-dimethylamino-1-(2-thienyl)-1-propanone.

10. The process of claim 5, wherein the mixture is maintained at about room temperature to obtain the N,N-dimethyl-3-(2-thienyl)-3-hydroxypropanamine borane.

11. The process of claim 5, wherein the mixture is maintained for at least about 5 minutes to obtain the N,N-dimethyl-3-(2-thienyl)-3-hydroxypropanamine borane.

12. The process of claim 11, wherein the mixture is maintained for about 20 minutes to about 5 hours.

13. The process of claim 11, wherein the mixture is maintained for about one hour.

14. The process of claim 5, wherein the BH₃ is added to the 3-dimethylamino-1-(2-thienyl)-1-propanone and the polar aprotic solvent in the form of B₂H₆ or a complex with a Lewis base.

15. The process of claim 14, wherein the complex with the Lewis base is a borane tetrahydrofuran complex, borane 1,2-bis(tert-butylthio)ethane complex, borane 1,4-oxathiane complex, borane 4-methylmorpholine complex, borane ammonia complex, borane dimethyl sulfide complex, borane dimethylamine complex, borane diphenylphosphine complex, borane isoamylsulfide complex, borane morpholine complex, borane-morpholine, borane N,N-diethylaniline complex, borane N,N, diisopropylethylamine complex, borane pyridine complex, borane tert-butylamine complex, borane triethylamine complex, borane trimethylamine complex, or borane triphenylphosphine complex.

16. The process of claim 5, wherein the N,N-dimethyl-3-(2-thienyl)-3-hydroxypropanamine borane is (S)—N,N-dimethyl-3-(2-thienyl)-3-hydroxypropanamine borane.

17. The process of claim 5, wherein the N,N-dimethyl-3-(2-thienyl)-3-hydroxypropanamine borane is (R)—N,N-dimethyl-3-(2-thienyl)-3-hydroxypropanamine borane.

18. A process for preparing duloxetine or a pharmaceutically acceptable salt of duloxetine comprising:
(a) preparing (S)—N,N-dimethyl-3-(2-thienyl)-3-hydroxypropanamine borane by the process of claim 5; and
(b) converting the (S)—N,N-dimethyl-3-(2-thienyl)-3-hydroxypropanamine borane into duloxetine or a pharmaceutically acceptable salt of duloxetine.

19. The process of claim 18, wherein the chiral oxazaborolidine catalyst is an (R)-oxazaborolidine catalyst.

20. The process of claim 18, wherein the pharmaceutically acceptable salt of duloxetine is a hydrochloride, oxalate, phosphate, succinate, fumarate, benzenesulfonate, maleate, or tartarate salt.

21. The process of claim 18, wherein the pharmaceutically acceptable salt of duloxetine is a hydrochloride salt.

22. A process for preparing N,N-dimethyl-3-(1-naphthalenyloxy)-3-(2-thienyl)propanamine or a salt thereof comprising:
(a) combining a base, N,N-dimethyl-3-(2-thienyl)-3-hydroxypropanamine borane, and at least one polar aprotic solvent to obtain a solution;
(b) combining the solution with a 1-halonaphthalene to obtain a mixture;
(c) heating the mixture to obtain N,N-dimethyl-3-(1-naphthalenyloxy)-3-(2-thienyl)propanamine; and, optionally
(d) converting the N,N-dimethyl-3-(1-naphthalenyloxy)-3-(2-thienyl)propanamine to a salt thereof.

23. The process of claim 22, wherein the salt of N,N-dimethyl-3-(1-naphthalenyloxy)-3-(2-thienyl)propanamine is a hydrochloride, oxalate, phosphate, succinate, fumarate, benzenesulfonate, maleate, or tartarate salt.

24. The process of claim 22, wherein the salt of N,N-dimethyl-3-(1-naphthalenyloxy)-3-(2-thienyl)propanamine is a maleate salt.

25. The process of claim 22, wherein the base is an alkali metal hydroxide or alkali metal alkoxide.

26. The process of claim 22, wherein the base is potassium hydroxide, sodium methoxide, or sodium hydroxide.

27. The process of claim 22, wherein the polar aprotic solvent is a C₅-C₈ aromatic hydrocarbon, ionic liquid, dimethyl sulfoxide, dimethylformamide, dimethylacetamide, acetonitrile, sulfolane, nitromethane or propylene carbonate.

28. The process of claim 27, wherein the C₅-C₈ aromatic hydrocarbon is toluene or xylene.

29. The process of claim 27, wherein the ionic liquid is a alkylammonium halide, alkylphosphonium halide, N-alkylpyridinium halide, N-N-dialkylimidazolium halide, tetraalkylammonium tetraalkylboride, 1-alkyl-3-methylimidazolium trifluoromethanesulfonate salt, monoalkylammonium nitrate salt, halogenaluminate, chlorocuprate or 1-butyl-3-methylimidazolium tetrafluoroborate.

30. The process of claim 27, wherein the ionic liquid is 1-butyl-3-methylimidazolium tetrafluoroborate.

31. The process of claim 22, wherein the polar aprotic solvent is dimethylacetamide or dimethylsulfoxide.

32. The process of claim 22, wherein the combination of step a) is maintained at a temperature of from about 15° C. to about reflux temperature of the polar aprotic solvent to obtain the solution.

33. The process of claim 22, wherein the 1-halonaphthalene is 1-fluoronaphthalene or 1-chloronaphthalene.

34. The process of claim 22, wherein the mixture is cooled to room temperature before combining with the 1-halonaphthalene.

35. The process of claim 22, wherein the mixture is heated to a temperature of about room temperature to about reflux temperature of the polar aprotic solvent to obtain the N,N-dimethyl-3-(1-naphthalenyloxy)-3-(2-thienyl)propanamine.

36. The process of claim 24, wherein the N,N-dimethyl-3-(1-naphthalenyloxy)-3-(2-thienyl)propanamine is converted to N,N-dimethyl-3-(1-naphthalenyloxy)-3-(2-thienyl)propanamine maleate by a process comprising:
(a) combining with maleic acid a solution of N,N-dimethyl-3-(1-naphthalenyloxy)-3-(2-thienyl)propanamine in at least one solvent to obtain a precipitate of N,N-dimethyl-3-(1-naphthalenyloxy)-3-(2-thienyl)propanamine maleate; and
(b) recovering the N,N-dimethyl-3-(1-naphthalenyloxy)-3-(2-thienyl)propanamine maleate.

37. The process of claim 36, wherein the solvent is a C ₁₋₈ alcohol, C₃₋₇ ester, C₃₋₈ ether, C₃₋₇ ketone, C₆₋₁₂ aromatic hydrocarbon, acetonitrile, or water.

38. The process of claim 36, wherein the solvent is acetone, n-butanol, ethyl acetate, methyl tert-butyl ether, toluene or water.

39. The process of claim 36, wherein the solvent is ethyl acetate, acetone, or n-butanol.

40. The process of claim 36, wherein the combination of N,N-dimethyl-3-(1-naphthalenyloxy)-3-(2-thienyl)propanamine, maleic acid, and solvent is heated.

41. The process of claim 40, wherein the combination is heated to about reflux temperature of the solvent.

42. The process of claim 36, wherein the combination of N,N-dimethyl-3-(1-naphthalenyloxy)-3-(2-thienyl)propanamine, maleic acid, and solvent is cooled to induce precipitation of the N,N-dimethyl-3-(1-naphthalenyloxy)-3-(2-thienyl)propanamine maleate.

43. The process of claim 42, wherein the wherein the combination of N,N-dimethyl-3-(1-naphthalenyloxy)-3-(2-thienyl)propanamine, maleic acid, and solvent is cooled to a temperature of about 15° C.

44. The process of claim 42, wherein the combination of N,N-dimethyl-3-(1-naphthalenyloxy)-3-(2-thienyl)propanamine, maleic acid, and solvent is maintained, while cooling, for about 20 minutes to about 5 days.

45. The process of claim 36, wherein the N,N-dimethyl-3-(1-naphthalenyloxy)-3-(2-thienyl)propanamine maleate is (S)—N,N-dimethyl-3-(1-naphthalenyloxy)-3-(2-thienyl)propanamine maleate.

46. The process of claim 45, wherein the (S)—N,N-dimethyl-3-(1-naphthalenyloxy)-3-(2-thienyl)propanamine maleate has less than about 5% (R)—N,N-dimethyl-3-(1-naphthalenyloxy)-3-(2-thienyl)propanamine maleate.

47. The process of claim 45, wherein the (S)—N,N-dimethyl-3-(1-naphthalenyloxy)-3-(2-thienyl)propanamine maleate has less than about 2% (R)—N,N-dimethyl-3-(1-naphthalenyloxy)-3-(2-thienyl)propanamine maleate.

48. The process of claim 45, wherein the (S)—N,N-dimethyl-3-(1-naphthalenyloxy)-3-(2-thienyl)propanamine maleate has about 1% (R)—N,N-dimethyl-3-(1-naphthalenyloxy)-3-(2-thienyl)propanamine maleate.

49. A process for preparing duloxetine or a pharmaceutically acceptable salt of duloxetine comprising:
(a) preparing (S)—N,N-dimethyl-3-(1-naphthalenyloxy)-3-(2-thienyl)propanamine maleate by the process of claim 22; and
(b) converting the (S)—N,N-dimethyl-3-(1-naphthalenyloxy)-3-(2-thienyl)propanamine maleate into duloxetine or a pharmaceutically acceptable salt of duloxetine.

50. The process of claim 49, wherein the pharmaceutically acceptable salt of duloxetine is a hydrochloride, oxalate, phosphate, succinate, fumarate, benzenesulfonate, maleate, or tartarate salt.

51. The process of claim 49, wherein the pharmaceutically acceptable salt of duloxetine is a hydrochloride salt.

52. A one-pot process for preparing N,N-dimethyl-3-(1-naphthalenyloxy)-3-(2-thienyl)propanamine or a acceptable salt thereof comprising:
(a) combining 3-dimethylamino-1-(2-thienyl)-1-propanone, at least one polar aprotic solvent, a chiral oxazaborolidine catalyst and BH₃ to obtain a first mixture containing N,N-dimethyl-3-(2-thienyl)-3-hydroxypropanamine borane;
(b) combining the first mixture with a base and at least one polar aprotic solvent to obtain a solution;
(c) combining the solution with a 1-halonaphthalene to obtain a second mixture;
(d) heating the second mixture to obtain N,N-dimethyl-3-(1-naphthalenyloxy)-3-(2-thienyl)propanamine; and, optionally
(e) converting the N,N-dimethyl-3-(1-naphthalenyloxy)-3-(2-thienyl)propanamine to a salt thereof.

53. The process of claim 52, wherein the salt of N,N-dimethyl-3-(1-naphthalenyloxy)-3-(2-thienyl)propanamine is a hydrochloride, oxalate, phosphate, succinate, fumarate, benzenesulfonate, maleate, or tartarate salt.

54. The process of claim 52, wherein the salt of N,N-dimethyl-3-(1-naphthalenyloxy)-3-(2-thienyl)propanamine is a maleate salt.
